(19) United States
(12) Patent Application Publication
(10) Pub. No.: US 2002/0119909 A1
(43) Pub. Date: Aug. 29, 2002

(54) Preparation of enantiomerically enriched flavor and fragrance components

(75) Inventors: Roger B. Pettman, Weston, MA (US); Richard A. Silva, Foxborough, MA (US); Jay Francis Larrow, Wakefield, MA (US); Isabelle Storet, Les Eparres (FR)

Correspondence Address: Paul D. Greeley, Esq., Ohlandt, Greeley, Ruggiero & Perle, L.L.P., 10th Floor, One Landmark Square, Stamford, CT, 06901-2682, US

(73) Assignee: Rhodia ChiRex, Inc.

(21) Appl. No.: 10/051,771

(22) Filed: Jan. 17, 2002

Related U.S. Application Data
(60) Provisional application No. 60/262,714, filed on Jan. 19, 2001.
(60) Provisional application No. 60/293,408, filed on May 24, 2001.
(60) Provisional application No. 60/340,166, filed on Dec. 14, 2001.

(51) Int. Cl. A61K 7/46; C07C 29/10

(52) U.S. Cl. 512/25; 568/867

(57) ABSTRACT

The present invention includes a process for enantioselective preparation of a non-racemic compound, which is either usable as a fragrance or flavor component or is convertible to a fragrance or flavor component by one or more additional reaction steps. The process includes the step of contacting either a substrate capable of forming a non-racemic compound by an enantioselective reaction and a co-reactant in the presence of a non-racemic catalyst, or a non-racemic or enantiopure substrate and a co-reactant, optionally in the presence of a racemic or non-racemic catalyst. The contacting is carried out at a temperature and length of time that is sufficient to produce the non-racemic compound with high optical purity. The process is used in stereoselective preparation of enantiomerically enriched intermediates useful in the preparation of non-racemic, chiral flavor and fragrance components.

This application claims priority from U.S. Provisional Application Serial No. 60/262,714, filed Jan. 19, 2001, U.S. Provisional Application Serial No. 60/293,408, filed May 24, 2001 and U.S. Provisional Application Serial No. 60/340,166, filed Dec. 14, 2001.

### BACKGROUND OF THE INVENTION

1. FIELD OF THE INVENTION

The present invention relates to a process for enantioselective preparation of non-racemic compounds which are usable as fragrance or flavor components or can be converted to a fragrance or flavor component. More particularly, the present invention relates to a process for the preparation of a non-racemic compound by enantioselective hydrogenation, hydroboration, hydride transfer, alkylation, vinylation, epoxidation, epoxide ring opening, acetalization, ketalization, acylation, nucleophilic substitution or a combination thereof.

2. DESCRIPTION OF THE PRIOR ART

The demand for enantiomerically pure compounds or non-racemic compounds having high optical purity, i.e., having an optical purity of at least 85% enantiomeric excess, has grown rapidly in recent years. The impetus for rapid growth has been the potential uses of such compounds as biologically active compounds or as intermediates in the synthesis of such biologically active compounds, particularly in the pharmaceutical and agrochemical industries.

It has become increasingly clear that enantiomerically pure drugs have numerous advantages over racemic drug mixtures including advantages, such as, fewer side effects and greater potency, which result in part from the ability of living systems to differentiate between enantiomeric compounds. Some of these advantages are summarized in a review article in Chem. Eng. News, pp. 46-79, Sep. 28, 1992.

In the case of flavors and fragrances, many natural, non-racemic compounds have different olfactory properties. For example, d-carvone (A) has the odor (flavor) of caraway, while the enantiomeric l-carvone (B) has the odor (flavor) of spearmint.

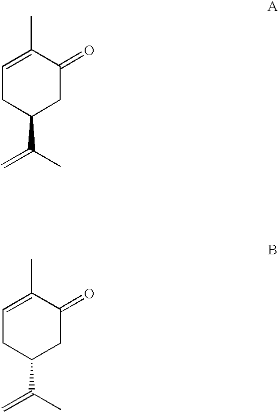

While many natural products have been used as sources of flavor and fragrance components, the pool of potential products is limited to certain structures and configurations found in nature.

In the manufacture of synthetic products, traditional methods of organic synthesis have often been designed and optimized for the production of racemic materials. Thus, non-natural chiral compounds have been developed and used as flavors and fragrances primarily as racemic mixtures, which have equal parts of each enantiomeric form and, as a result, exhibit certain fragrance or flavor properties characteristic to the racemic mixture.

Because each enantiomer can potentially have different olfactory properties, a racemic mixture can have unpleasant or unbalanced characteristics relative to enantiomerically pure or enantiomerically enriched preparations. To overcome this disadvantage, laborious procedures and separations would be required to either enrich the racemic product in one or the other enantiomer or to completely separate one enantiomer from the other. The present invention overcomes the above disadvantages by providing a process which enables one to obtain an enantiomerically pure or enantiomerically enriched product directly, without the need for laborious procedures and separations.

### SUMMARY OF THE INVENTION

The present invention includes a process for enantioselective preparation of a non-racemic compound usable as a fragrance or flavor component or is convertible to a fragrance or flavor component. The process comprises contacting: (a) a substrate capable of forming a non-racemic compound by an enantioselective reaction and at least one co-reactant in the presence of a non-racemic catalyst; or (b) a non-racemic or enantiopure substrate and at least one co-reactant, optionally in the presence of a racemic or non-racemic catalyst. The contacting is carried out at a temperature and for a length of time sufficient to produce the non-racemic compound in high optical purity.

The present invention further includes a non-racemic compound prepared by the process of the present invention.

The process of the present invention enables one to obtain an enantiomerically pure or enantiomerically enriched product directly, without the need for laborious procedures and separations and to obtain the most desirable fragrance or flavor formulation by balancing the amounts of each enantiomer to optimize a particular desirable fragrance or flavor property.

In another aspect, the present invention is directed to a non-racemic composition comprising an enantiopure enantiomer of structural formula (1) or (2), or a non-racemic mixture of an enantiomer of structural formula (1) and an enantiomer of structural formula (2):

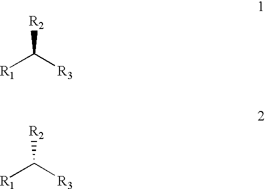

wherein:

R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;

R′ is (C₁-C₄)alkylene;

R″ is (C₁-C₁₂)alkyl, (C₆-C₁₂)aryl, (C₇-C₁₄)aralkyl, (C₇-C₁₄)alkaryl or (C₁-C₁₂)carboxylate; and

R₂ is hydroxy or (C₁-C₁₂)carboxylate.

In another aspect, the present invention is directed to a process for making a perfuming composition or perfumed article, comprising adding to such composition, or article an effective quantity of a non-racemic composition comprising an enantiopure enantiomer of structural formula (1) or (2), or a non-racemic mixture of an enantiomer of structural formula (1) and an enantiomer of structural formula (2):

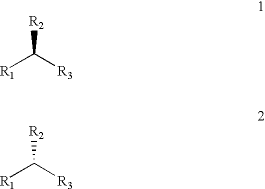

wherein:

R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;

R′ is (C₁-C₄)alkylene;

R″ is (C₁-C₁₂)alkyl, (C₆-C₁₂)aryl, (C₇-C₁₄)aralkyl, (C₇-C₁₄)alkaryl or (C₁-C₁₂)carboxylate; and

R₂ is hydroxy or (C₁-C₁₂)carboxylate.

In another aspect, the present invention is directed to a perfuming composition or perfumed article comprising, in an amount effective to influence the odor of the composition or article, a non-racemic composition comprising an enantiopure enantiomer of structural formula (1) or (2), or a non-racemic mixture of an enantiomer of structural formula (1) and an enantiomer of structural formula (2):

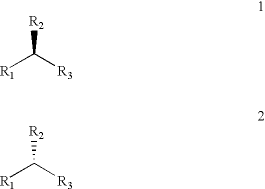

wherein:

R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;

R′ is (C₁-C₄)alkylene;

R″ is (C₁-C₁₂)alkyl, (C₆-C₁₂)aryl, (C₇-C₁₄)aralkyl, (C₇-C₁₄)alkaryl or (C₁-C₁₂)carboxylate; and

R₂ is hydroxy or (C₁-C₁₂)carboxylate.

### DETAILED DESCRIPTION OF THE INVENTION

The term “non-racemic” in the context of the present invention includes any enantiomerically enriched compound, including enantiopure compounds, but excluding racemic mixtures.

The expression “substrate capable of forming a non-racemic compound by an enantioselective reaction”, as used in the context of the present invention, includes prochiral substrates and racemic substrates. An example of such a prochiral substrate is the olefin 14a (R₁=phenyl; R₂=methyl; R₃═H; R₄═CO₂Et) which can be enantioselectively epoxidized to the (R)- or (S)-epoxide 13a (below).

An example of such a racemic substrate is a racemic epoxide, which can produce an enantiomerically enriched 1,2-diol and an enantiomerically enriched unreacted epoxide upon kinetic resolution, i.e., upon selectively reacting one enantiomer with water.

Hydrolytic kinetic resolution (HKR) technology, which involves a stereoselective reaction of a nucleophile, such as, water with a racemic, chiral epoxide, in the presence of a non-racemic catalyst is disclosed U.S. Pat. Nos. 5,665,890 and 5,929,232 to Jacobsen et al. The contents of these patents are incorporated herein by reference as if fully set forth. For example, according to this technology, kinetic resolution of a racemic mixture can be achieved by selectively reacting one enantiomer, thereby producing an enantiomerically enriched 1,2-diol product and an enantiomerically enriched unreacted epoxide, both of which can be isolated in a high enantiomeric excess. Asymmetric epoxidation of prochiral olefins with bleach in the presence of a non-racemic catalyst to yield enantiomerically enriched epoxides is described in U.S. Pat. No. 5,627,739, also to Jacobsen et al., the contents of which are incorporated herein by reference as if fully set forth. The references do not teach the preparation of enantiomerically pure or enantiomerically enriched flavors and fragrances or precursors thereof. Accordingly, an object of the present is to provide a practical and economical method of preparing enantiomerically pure or enantiomerically enriched compounds, which can be used as flavors and fragrances or as intermediates in the stereoselective synthesis of chiral flavors and fragrances.

In one embodiment, the process includes the step of contacting a substrate, which is capable of forming a non-racemic compound by an enantioselective reaction, and one or more co-reactants in the presence of a non-racemic catalyst, under reaction conditions, such as, reaction temperature and reaction time that produce a non-racemic compound.

In another embodiment, the process includes the step of contacting a non-racemic or enantiopure substrate and one or more co-reactant, optionally in the presence of a racemic or non-racemic catalyst. As before, the contacting is carried out at a temperature and length of time that is sufficient to produce a non-racemic compound.

The present invention includes a novel non-racemic compound, as well as an enantioselective process for preparing a non-racemic compound, which can be an enantiopure single enantiomer or an enantiomerically enriched mixture of enantiomers represented by the below formulae, wherein the chiral centers are indicated by an asterisk. R₁*CHR₂R₃ are represented by the formulae 1 and 2:

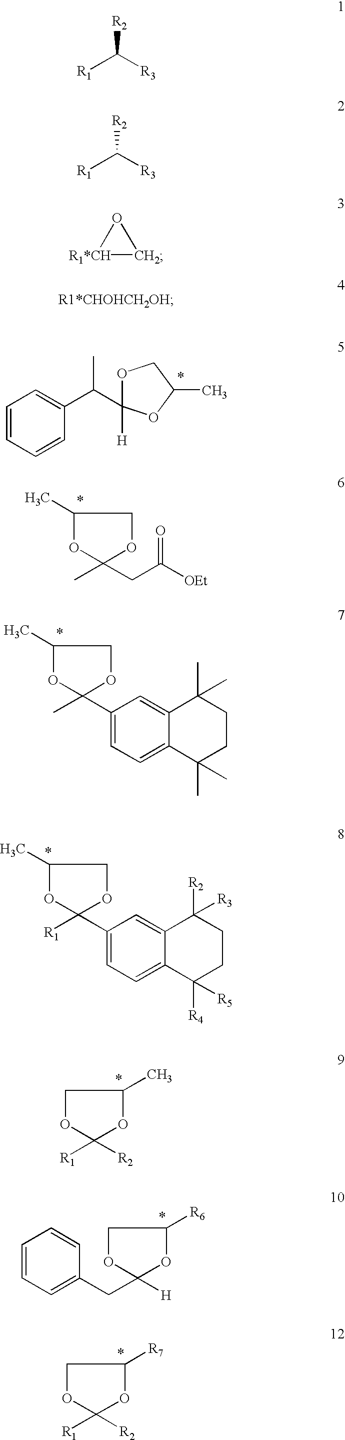

wherein in compounds 1 and 2, R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″; R′ is (C₁-C₄)alkylene; R″ is (C₁-C₁₂)alkyl, (C₆-C₁₂)aryl, (C₇-C₁₄)aralkyl, (C₇-C₁₄)alkaryl or (C₁-C₁₂)carboxylate; and R₂ is hydroxy or (C₁-C₁₂)carboxylate and in a preferred embodiment R₁ in compounds 1 and 2 can be a linear, branched or cyclic alkyl of 1 to 12 carbon atoms, aryl of 6 to 12 carbon atoms, aralkyl of 7 to 14 carbon atoms, alkaryl of 7 to 14 carbon atoms and ((1R, 2S, 5R)-(−)-menthoxy)CH₂; R₂ can be hydroxy and a carboxylate of 1 to 12 carbon atoms; and R₃ can be a linear, branched or cyclic alkyl of 1 to 12 carbon atoms, alkenyl of 1 to 12 carbon atoms, hydroxy(C₁-C₁₂)alkyl, more preferably hydroxymethyl and acyloxyalkyl, more preferably acyloxymethyl of 1 to 12 carbon atoms;

wherein R₁ in compounds 3 and 4 can be a linear, branched or cyclic alkyl of 1 to 12 carbon atoms, aryl of 6 to 12 carbon atoms, aralkyl of 7 to 14 carbon atoms, alkaryl of 7 to 14 carbon atoms, halo(C₁-C₆)alkyl, more preferably halomethyl, and ((1R, 2S, 5R)-(−)-menthoxy)CH₂;

wherein each R₁, R₂, R₃, R₄ and R₅ in compound 8 can independently be hydrogen, a linear, branched or cyclic alkyl of 1 to 12 carbon atoms, aryl of 6 to 12 carbon atoms, aralkyl of 7 to 14 carbon atoms and alkaryl of 7 to 14 carbon atoms;

wherein each R₁ and R₂ in compound 9 can independently be hydrogen, a linear, branched or cyclic alkyl of 1 to 12 carbon atoms, aryl of 6 to 12 carbon atoms, aralkyl of 7 to 14 carbon atoms and alkaryl of 7 to 14 carbon atoms;

wherein R₆ in compound 10 is hydroxymethyl;

wherein each R₁ and R₂ in compound 12 can be a linear, branched or cyclic alkyl of 1 to 12 carbon atoms, aryl of 6 to 12 carbon atoms, aralkyl of 7 to 14 carbon atoms and alkaryl of 7 to 14 carbon atoms; and R₇ can be a linear, branched or cyclic alkyl of 1 to 12 carbon atoms, aryl of 6 to 12 carbon atoms, heteroaryl of 6 to 12 carbon atoms, aralkyl of 7 to 14 carbon atoms and alkaryl of 7 to 14 carbon atoms; and

wherein R₁ in compound 13 is phenyl; R₂ is methyl; R₃ is hydrogen; and R₄ is CO₂Et.

As used herein, “alkyl” means a saturated linear, branched or cyclic hydrocarbon group, such as, for example, methyl ethyl, propyl, n-butyl, isobutyl, tert-butyl, hexyl, cyclohexyl, octyl, cyclooctyl, decyl, dodecyl, stearyl, eicosyl.

As used herein, “(C₁-C₄)alkylene” means a bivalent acyclic saturated hydrocarbon group containing from one to four carbon atoms per group and having its free bonds on two different atoms, such as, for example, methylene, dimethylene, trimethylene.

As used herein, “alkoxy” means an acyclic ether group according to the formula RO—, wherein R is alkyl, such as, for example, methoxy, ethoxy, propoxy.

As used herein, :hydroxyalkyl” means a hydroxy substituted alkyl group, such as, for example, hydroxymethyl, hydroxypropyl, hydroxybutyl.

As used herein, “alkenyl” means a linear, branched or cyclic hydrocarbon group containing one or more unsaturated sites, that is, carbon-carbon double bonds, per group, such as, for example, vinyl, allyl, isopropenyl, 2-methyl-2-propenyl, cyclohexadienyl.

As used herein, “aryl” means an unsaturated hydrocarbon ring system containing one or more aromatic rings per group, which in the case of an aryl group containing two or more rings per group are fused rings and which may be substituted on one or more aromatic carbon atoms with hydroxyl, (C₁-C₄)alkoxy, (C₁-C₆)carbonyl, —C(O)OH, carbo(C₁-C₄)alkoxy, (C₁-C₁₂)alkenyl or halo, such as, for example, phenyl, 2-naphthyl, 2-phenanthryl, 4-hydroxyphenyl, 2-methoxyphenyl, 4-formylphenyl, 4-carbomethoxyphenyl, 4-propenylphenyl, 4-chlorophenyl.

As used herein, the term “alkaryl” means an aromatic ring that is substituted on one or more carbon atoms of the aromatic ring by (C₁-C₄)alkyl, such as, for example, 4-methylphenyl, 2-isopropylphenyl, 2,6-dimethylphenyl.

As used herein, “aralkyl” means an alkyl group that is substituted on one of the alkyl carbon atoms by an aryl group and which may, optionally, be substituted on one or more carbon atoms of the aromatic ring of the aryl group with (C₁-C₄)alkyl, hydroxyl, (C₁-C₄)alkoxy, (C₁-C₆)carbonyl, —C(O)OH, carbo(C₁-C₄)alkoxy, (C₁-C₁₂)alkenyl or halo, such as for example, phenylmethyl, phenylethyl, ethylphenylmethyl, hydroxyphenylmethyl, methoxyphenylmethyl, 4-formylphenylmethyl, 4-carbomethoxyphenylethyl, propenylphenylmethyl, chlorophenylmethyl. The phenylmethyl group is at times referred to herein by the equivalent term “benzyl”.

As used herein, the term “carboxylate” means a RC(O)O— group, wherein R is alkyl, preferably (C₁-C₆)alkyl. Examples of the carboxylate include formate, acetate, propionate, and butyrate.

As used herein, the term “acyloxyalkyl” means an alkyl group, preferably (C₁-C₄)alkyl, that is substituted on one of the alkyl carbon atoms with a carboxylate group, preferably a (C₂-C₇)carboxylate group, such as, for example, —CH₂OC(O)CH₃, —CH₂OC(O)CH₂CH₃.

As used herein, the term “halo(C₁-C₆)alkyl” means a halo substituted alkyl group containing from one to six carbon atoms per group, such as for example, chloromethyl, bromomethyl, iodomethyl, fluoromethyl, chloroethyl, chloropropyl.

As used herein, use of the notation “(Cₙ-Cₘ)”, wherein n and m are each integers, in reference to an organic group means that such group contains from n carbon atoms to m carbon atoms per group.

As described above, the present invention includes a process for enantioselective preparation of a non-racemic compound, which is either usable as a fragrance or flavor component or is convertible to a fragrance or flavor component by one or more additional reaction steps. The non-racemic compound, which is the product of the process of the present invention, has an optical purity of at least 1% enantiomeric excess. Preferably, non-racemic compound has an optical purity of at least 75% enantiomeric excess, more preferably at least 95% enantiomeric excess, and most preferably, the non-racemic compound, which is the product of the process of the present invention, has an optical purity of at least 99% enantiomeric excess or is an enantiopure single enantiomer.

Preferably, the enantioselective reaction in the process of the present invention can be hydrogenation, hydroboration, hydride transfer, alkylation, vinylation, epoxidation, epoxide ring opening, acetalization, ketalization, acylation, nucleophilic substitution or a combination thereof. The process is suitable for use in stereoselective preparation of enantiomerically enriched intermediates useful in the preparation of non-racemic, chiral flavor and fragrance components.

Specific examples of enantiomerically pure fragrances or precursors synthesized using our invention include:

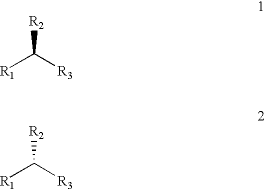

1a and 2a: R₁═CH₃(CH₂)₅; R₂═OH or acetate; R₃═CH₂CH₃.

1b and 2b: R₁=((1R, 2S, 5R)-(−)-menthoxy)CH₂; R₂═OH or acetate; R₃═CH₂OH.

1c and 2c: R₁=benzyl; R₂═OH; R₃═(CH₃)₂CHCH₂

1d and 2d: R₁═CH₃; R₂=isobutyrate; R₃═H₂C═C(CH₃)CH₂.

1e and 2e: R₁=4-methylphenyl; R₂═OH or acetate; R₃═CH₃.

1f and 2f: R₁=phenyl; R₂═OH or acetate; R₃═CH₂OAc.

1g and 2g: R₁═CH₃; R₂═OH; R₃═CH₂OH.

1h and 2h: R₁=phenyl; R₂═OH; R₃═CH₃.

1i and 2i: R₁=phenyl; R₂=acetate; R₃═CH₃.

1j and 2j: R₁=phenyl; R₂=propionate; R₃═CH₃.

1k and 2k: R₁═CH₂CH₃, R₂═OH or acetate; R₃═CH₃ (CH₂)₃.

1l and 2l: R₁═CH₃(CH₂)₄; R₂═OH or acetate; R₃═CH₃.

In a preferred embodiment, the non-racemic composition of the present invention comprises: (a) an enantiopure enantiomer of structural formula (1) or (2), or (b) a non-racemic mixture of an enantiomer of structural formula (1) and an enantiomer of structural formula (2), wherein:

R₁ is (C₁-C₁₂)alkyl, (C₆-C₁₂)aryl, (C₇-C₁₄)aralkyl, (C₇-C₁₄)alkaryl or ((1R, 2S, 5R)-(−)-menthoxy)CH₂, more preferably, C₇-C₁₄)alkaryl;

R₂ is hydroxy or (C₁-C₁₂)carboxylate; and

R₃ is (C₁-C₁₂)alkyl, (C₂-C₁₂)alkenyl, hydroxyalkyl or (C₁-C₁₂)acyloxymethyl, more preferably, (C₁-C₈)alkyl.

In an even more highly preferred embodiment, the non-racemic composition of the present invention comprises: (a) an enantiopure compound corresponding to structural formula (1c) or (2c) above, that is, an enantiopure compound according to structural formula (1) or (2) wherein R₁ is benzyl, R₂ is hydroxy and R₃ is isobutyl, or (b) a non-racemic mixture of a compound corresponding to structural formula (1c) and a compound corresponding to structural formula (2c).

It has been discovered that benzylisobutylcarbinol, that is, the enantiomeric compounds according to structural formulae (1c) or (2c), has a different odour depending on its optically active form. In its (S) form, benzylisobutylcarbinol has a particularly interesting floral-green, mimosa powdery note. The (R) form has a less natural green rose note.

In one embodiment, the non-racemic composition of the present invention consists essentially of, or, advantageously, consists of: (a) an enantiopure enantiomer of structural formula (1) or (2), or (b) a non-racemic mixture of an enantiomer of structural formula (1) and an enantiomer of structural formula (2).

The compounds mentioned above can be obtained in enantiomeric excesses greater than 98%, preferably in greater than 99.9%, using Hydrolytic Kinetic Resolution (HKR) methodology developed by Jacobsen in the previously incorporated U.S. Pat. Nos. 5,665,890 and 5,929,232. The resulting enantiomers can be mixed in varying ratios ranging from R/(S+R)×100=99.999999% to S/(S+R)×100=99.999999. Racemic mixtures are excluded.

The process for the general enantioselective synthesis of fragrances 1 and 2 begins with the Hydrolytic Kinetic Resolution of epoxides of the general structure 3, where R₁ can be a cyclic or straight alkyl group, with or without appended functionality. R₁ can also be aryl, with or without appended functional groups.

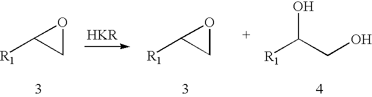

Specific examples of epoxides resolved by the process of the present invention include:

3a or 4a is R₁═CH₃(CH₂)₅;

3b or 4b is R₁═ClCH₂;

3c or 4c is R₁═(CH₃)₂CHCH₂;

3d or 4d is R₁=phenyl;

3e or 4e is R₁=4-methylphenyl;

3f or 4f is R₁═CH₃(CH₂)₃; and

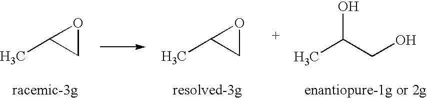

In this case, the use (R, R)-Cobalt Salen catalyst described by Jacobsen gave the corresponding (R)-epoxide and the (S)-diol, except in the case of 3b where the order was reversed. The use of (S, S)-Cobalt Salen catalyst gave the corresponding (S)-epoxide and the (R)-diol, except in the case of 3b where the order was reversed.

If desired, the epoxides can be purified by distillation, recrystallization, otherwise, they can be used “as is”, without further purification. Similarly, the diols can be purified by distillation, recrystallization or used “as is”.

The term “as is” refers to using the epoxide or the diol obtained from a kinetic resolution process in a telescoped fashion by using the reaction mixture as the starting material after removal of the catalyst from the diol and the epoxide.

Thus, fragrances 1a and 2a can be obtained by allowing the appropriate Grignard (J. Org. Chem. 1987, 52, 4505) or alkyl lithium (J. Org. Chem. 1987, 52, 4505) reagent to react with the appropriate epoxide followed by derivatization with the appropriate anhydride or acid halide. Grignard and alkyl lithium reagents can be modified by protocols known in the art, i.e., treatment with metals, metal halides (for Grignard see: J. Org. Chem. 1989, 54, 1295; for alkyl lithium see: J. Amer. Chem. Soc., 1987, 109, 8105) or halo boranes (J. Amer. Chem. Soc., 1984, 106, 3693).

Compound 1b and 2b can be obtained by treatment of the acetonide, or any other standard diol protecting functionality, of 4b with (1R, 2S, 5R)-(−)-menthol in the presence of NaH or any other standard base (see “The Chemistry of the Ether Linkage,” ED. S. Patai, Interscience, New York (1967), pp. 445-498).

Fragrances 1 (h, i, and j), and 2 (h, i, and j) can be obtained by the regioselective reduction of epoxide 3d, after Hydrolytic Kinetic Resolution (HKR) followed by condensation with the appropriate anhydride or acid chloride. In this instance, the regioselective reduction of epoxides can be carried out using lithium tert-butylamine borane (J. Org. Chem., 1994, 59, 6378) or lithium aluminum hydride (J. Org. Chem. 1993, 58, 4727).

Fragrance compounds of formulae 1 and 2 according to the preferred embodiment wherein R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″; R′ is (C₁-C₄)alkylene; R″ is (C₁-C₁₂)alkyl, (C₆-C₁₂)aryl, (C₇-C₁₄)aralkyl, (C₇-C₁₄)alkaryl or (C₁-C₁₂)carboxylate; and R₂ is hydroxy or (C₁-C₁₂)carboxylate can be prepared by hydrolytic kinetic resolution of a racemic epoxide to give a resolved epoxide enatiomer, followed by ring opening of the resolved epoxide enantiomer with a nucleophile to form the fragrance compound.

A suitable racemic epoxide can be obtained reacting a peroxyacid acid, such as, for example, meta-chloroperbenzoic acid or peracetic acid, with an unsaturated compound, for example a compound of the formula R₂₁CH₂R₂₂, wherein R₂₁ is (C₁-C₁₂)alkyl, (C₁-C₆)aryl, (C₇-C₁₄)aralkyl, or (C₇-C₁₄)alkaryl and R₂₂ is (C₁-C₆)alkenyl.

In a preferred embodiment, the unsaturated compound is one according to the formula:

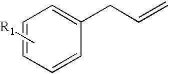

wherein R₁ is hydrogen, (C₁-C₄)alkyl or (C₁-C₄)alkoxy.

A method for preparing such a racemic epoxide is known and has been described in particular by Jerry March in “Advanced Organic Chemistry”, 4^{th} edition, John Wiley & Sons, 1992, p. 823. The reaction is carried out at a temperature of 25° C. to 50° C., in an organic solvent such as an aliphatic hydrocarbon, preferably halogenated, such as chloroform, dichloromethane or dichloroethane. At the end of the reaction, filtering is carried out to eliminate the solid residues, then extraction using a suitable organic solvent, for example tert-butyl ether. After washing the organic phase and concentrating, the epoxy compound obtained is recovered conventionally, for example by distillation. The epoxy compound obtained is in the racemic form.

In a preferred embodiment, the racemic epoxide is formed from the preferred unsaturated compound described above and is of the formula:

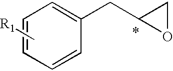

wherein R₁ is hydrogen, (C₁-C₄)alkyl or (C₁-C₄)alkoxy.

The racemic epoxide mixture is split by kinetic resolution by hydrolysis to produce one enantiomer in the form of a di-alcohol and the other enantiomer in the epoxy form. To this end, the racemic epoxy mixture is reacted with a nucleophile such as water in the presence of a optically active catalyst. The catalyst has been described in U.S. Pat. Nos. 5,665,890 and 5,929,232. Preferably, it is a complex between a transition metal, preferably Cr, Mn, V, Fe, Mo, W, Ru, Ni or Co, and the “Salen” ligand with the following formula:

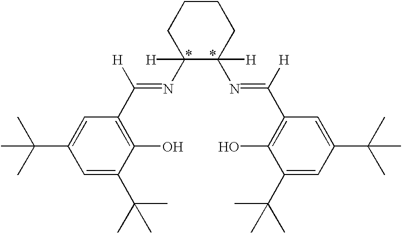

The catalyst is prepared using a transition metal, preferably cobalt and the ligand defined above obtained using the procedure described in Example 4 of U.S. Pat. No. 5,665,890. It is also possible to use the ligands described in U.S. Pat. No. 5,665,890 in which the cyclohexane-1,2-diyl is replaced by structures:

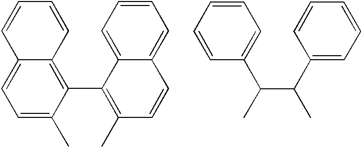

Preferably, it is (R,R)-Co-Salen which is prepared using a cobalt salt and the ligand (R,R)-N,N′-bis(3,5-di-tertbutylsalicylidene)-1,2-cyclohexanediamine. In the process of the invention, the racemic epoxy mixture is resolved by carrying out hydrolysis in the presence of the catalysts described above. The reaction is carried out between −20° C. and 50° C., preferably in the range 0° C. to ambient temperature (usually in the range 15° C. to 25° C.). It is advantageously carried out in a solvent preferably selected from tert-butylmethylether, ethyl ether or tetrahydrofuran. In one implementation of the invention, water is slowly added to the reaction medium comprising the racemic epoxide; the catalyst is the organic solvent. The resolved epoxide enantiomer is recovered conventionally, for example by distillation.

The resolved epoxy enantiomer is then reacted with a nucleophile, such as for example, a magnesium halide compound of the formula:

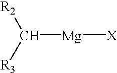

wherein R₂ and R₃, are each independently H, (C₁-C₁₂)alkyl, (C₂-C₁₂)alkenyl or (C₁-C₁₂)alkoxyalkyl and X is halo.

As used herein, “alkoxyalkyl” means an alkyl group that is substituted on one of the carbon atoms by an alkoxy group.

The ratio between the number of moles of magnesium halide and the number of moles of chiral epoxy compound is advantageously in the range 1 to 1.2. The reaction is carried out in the presence of a conventional catalyst, such as copper iodide or iodine. The quantity of catalyst, expressed with respect to the magnesium halide, represents 1 mole % to 10 mole %. The reaction is advantageously carried out at a temperature in the range of from −70° C. to 0° C. At the end of the reaction, an aqueous ammonium chloride solution is added to stop the reaction.

The aqueous and organic phases are separated. A conventional treatment is then carried out: washing the organic phase with brine (NaCl), then concentration of the organic phase. The optically active product fragrance molecule is then recovered from the organic phase obtained conventionally, for example by distillation.

If embodiments of such fragrance molecules wherein R₂ is C₁-C₁₂)carboxylate can be readily prepared from the alcohol by acylation with an acid halide or acid anhydride.

Products of particular interest, that is benzylisobutylcarbinol compounds of formulae 1c and 2c, are obtained by reaction of propenylbenzene with a peracid to form racemic (2,3-epoxypropyl)benzene, hydrolytic kinetic resolution of the racemic epoxide to the desired (2,3-epoxypropyl)benzene enantiomer and reaction of the (2,3-epoxypropyl)benzene enantiomer with an isopropylmagnesium halide to form the desired benzylisobutylcarbinol enantiomer.

The present invention also includes the enantioselective synthesis of ketals by the separate condensation of enantiomerically pure R and S propylene glycol (1g and 2g) with ketones, aldehydes and ketals.

Specific examples of these fragrances are shown below (in compound 10, R₆═CH₂OH). The asterisks in the ketalization reactions represent a chiral center having greater than 94% purity.

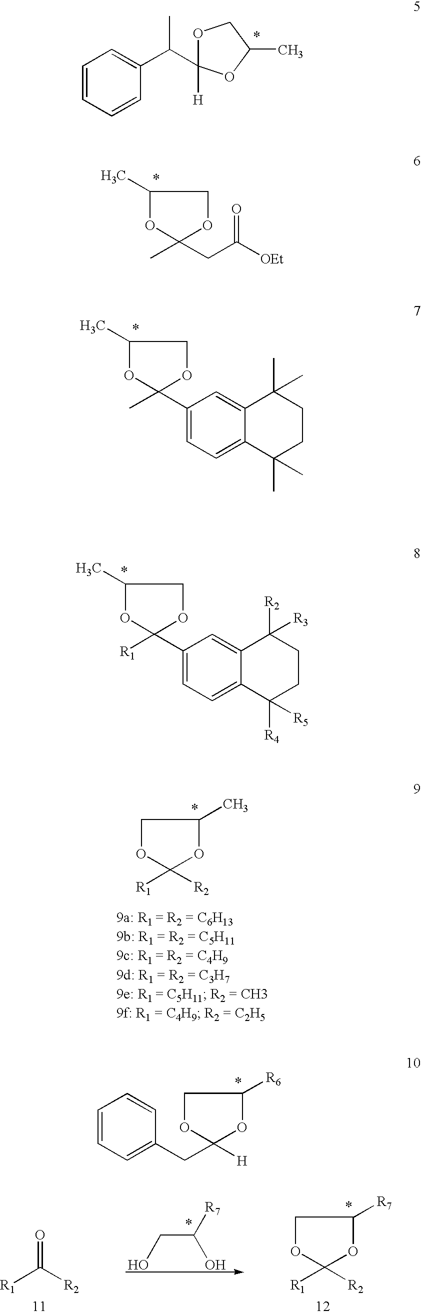

Preferably, for ketones and aldehydes of the structure 11 shown above, R₁, and R₂ can be hydrogen, cyclic or straight chain alkyl groups, with or without appended functionality, or aryl, with or without appended functional groups. Ketals can be synthesized using enantiomerically pure diols where R₇ can be alkyl, straight chain or cyclic, aryl, substituted or unsubstituted, or a heteroaryl group.

In ketones and aldehydes precursors of the structure 8, R₁, R₂, R₃, R₄ and R₅ can independently be hydrogen, cyclic or straight chain alkyl groups, or aryl, with or without appended functional groups.

In a preferred embodiment, non-racemic compound according to structure 7 or 8 is prepared by hydrolytic kinetic resolution of a racemic epoxide to give a non-racemic epoxide and an non-racemic diol, followed by condensation of the non-racemic diol with a ketone or aldehyde to form the non-racemic compound according to structure 7 or 8.

In a preferred embodiment, the non-racemic diol is condensed with a ketone or aldehyde according to the structural formula:

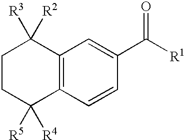

wherein R¹, R², R³, R⁴ and R⁵ are each independently H, alkyl or aryl. In one highly preferred embodiment, (R)-propane diol is condensed with a ketone according to the above structural formula, wherein R¹, R², R³, R⁴ and R⁵ are each methyl, to form (R)-Okoumal. In an alternative highly preferred embodiment, (S)-propane diol is condensed with a ketone according to the above structural formula, wherein R¹, R², R³, R⁴ and R⁵ are each methyl, to form (S)-Okoumal.

Fragrance 10 can be synthesized by allowing aldehyde 11a (R₁=benzyl; R₂═H) to condense with 4a or 4b followed by displacement of Cl with NaOH or KOH under phase transfer condition.

Fragrances 6, 7 and 9 can be obtained in greater than 98% ee, and in some cases in about 99.9% ee, by allowing the corresponding ketones, aldehydes or ketals to react with R or S propylene glycol (1g or 2g).

Compounds obtained from 5, 6, 7, 8, 9(e), 9(f) and 10 are obtained as a mixture of diastereomers provided that the chiral center marked with an asterisk has at least 90% ee.

The above-mentioned enantiomers and diastereomers can be combined in varying proportions to produce ratios ranging from R/(S+R)×100=99.999999% to S/(S+R)×100=99.999999. Racemic mixtures are excluded. The same is applicable to mixtures of 5 and 10.

The present invention further includes enantioselective synthesis of fragrances via enantioselective epoxidation using (R, R) or (S, S)-Mn Salen catalysts described by Jacobsen.

Preferably, alkenes can be enantioselectively synthesized, for example, where R₁ is phenyl, R₂ is alkyl, straight chain or cyclic, R₃ is hydrogen, carboxylic, ketonic, aldehydic, and R₄ is hydrogen, carboxylic, ketonic, or aldehydic.

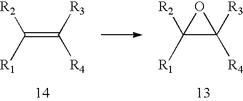

Thus, fragrance (R, R) or (S, S) 13a (R₁=phenyl; R₂=methyl; R₃═H; R₄═CO₂Et) can be synthesized enantioselectively by treatment of 14a (R₁=phenyl; R₂=methyl; R₃═H; R₄═CO₂Et) with either (R, R) or (S, S)-Mn Salen catalysts. In this case, the (R, R)-Mn Salen catalyst gives the (R, R) epoxide and the (S, S)-Mn Salen catalyst gives the (S, S) epoxide . Epoxides can be obtained in greater than 80% ee. If desired, recrystallization affords greater than 99% ee material. The resulting enantiomers can be mixed, with one another, in varying proportions to produce ratios ranging from R/(S+R)×100=99.999999% to S/(S+R)×100=99.999999. As before, the racemic mixtures are not included.

The isomeric fragrance (R, S) or (S, R) 13b in which the positions of the phenyl and methyl groups are interchanged, i.e., (R₁=methyl; R₂=phenyl; R₃═H; R₄═CO₂Et), can be synthesized enantioselectively by treatment of 14b (R₁=methyl; R₂=phenyl; R₃═H; R₄═CO₂Et) with either (R, R) or (S, S)-Mn Salen catalysts. In this case, the (R, R)-Mn Salen catalyst gives the (R, S) epoxide and the (S, S)-Mn Salen catalyst gives the (S, R) epoxide. Epoxides can be obtained in greater than 80% ee. As before, recrystallization affords greater than 99% ee material.

Non-racemic flavor and fragrance components prepared by the process of the present invention and their use as flavor or fragrance components in any ratio of enantiomers other than 50:50 (racemic) as well as the use of enantiomerically pure components in mixtures with other achiral or racemic components are contemplated by the present invention.

The products prepared by a process present invention can be used separately or as mixtures as perfume ingredients for the preparation of scented products that are suitable for use in perfumery. They can be formulated with a multitude of natural and synthetic products including alcohols, aldehydes, ketones, esters, lactones, acetals, solvents and diluents, and various other components that are commonly used in perfumery, such as, indole, p-menthane-8-thiol-3-one, methyleugenol, eugenol, anethol, and the like. The percentages in which these derivatives are used may vary within wide limits ranging from a few parts per thousand up to a few percent, usually in a carrier that includes alcoholic extracts.

There are no restrictions regarding the type of formulations and the destination of the actual finished product, which can include eau de cologne, toilet water (eau de toilette), scented water, perfume, cream, shampoo, deodorant, soap, detergent powder, household cleaner or softener.

As used herein, “perfuming composition” and “perfumed article” denote a mixture of various ingredients such as solvents, solid or liquid supports, fixatives, various scenting compounds, etc., into which the non-racemic compositions of the present invention are incorporated, which are used to produce a variety of types of finished products, with the desired fragrance. Perfume bases constitute preferred examples of perfuming compositions in which the non-racemic compositions of the present invention can advantageously be used. Eau de toilette, after-shave lotion, perfume, soap, bath or shower gel or deodorant or antiperspirant in the form of sticks or lotions constitute examples of finished products or substances which the non-racemic compositions of the present invention endow with their original note. They can also be used in all types of shampoos and hair-care products. They can also perfume all types of talcs or powders. They can also be used in room sprays or any cleaning product.

A further example of compositions in which the non-racemic compositions of the present can advantageously be used is represented by the usual detergent compositions. Such compositions generally comprise one or more of the following ingredients: anionic, cationic or amphoteric surfactants, bleaching agents, optical brighteners, various fillers, and anti-redepositing agents. The nature of these various components is not critical and the non-racemic compositions of the present invention can be added to any type of detergent composition. They can be introduced into fabric softeners, in liquid form or into compositions deposited on a support, usually a non-woven support, for use in clothes dryers.

The odor influencing effective amount of a non-racemic composition of the present invention, expressed as the percentage by weight in the perfume composition or perfumed article under consideration, depends on the nature of the perfume composition or perfumed article (a base for a perfume or eau de toilette, for example) and the strength and nature of the desired influence of the non-racemic composition in the finished product. It is clear that in a perfume base the quantity of non-racemic composition of the present invention can be very high, for example over 50% by weight, and can attain 90% by weight while in a perfume, an eau de toilette or an after-shave lotion, this quantity can be below 50% by weight. In detergent compositions, in particular for domestic use, and in soaps, the quantity of non-racemic composition of the present invention can be of the order of 1% to 2%. It can also be used in perfumed shampoos in an amount of 0.5% to 2%, or to perfume any hair product. Thus the lower limit of the amount of non-racemic composition of the present invention can be that which causes a perceptible modification in the scent or fragrance or the note of the finished product. In some cases, this minimum amount can be of the order of 0.01% by weight. Clearly, quantities which are not included in the limits indicated above can be employed without departing from the scope of the invention.

### EXAMPLE 1

### Preparation of (S)-benzylisobutylcarbinol

(S)-benzylisobutylcarbinol was prepared by reacting of isopropylmagnesium chloride with(R)-(2,3-epoxypropyl)benzene obtained from allylbenzene).

### A. Preparation of (2,3-epoxypropyl)benzene

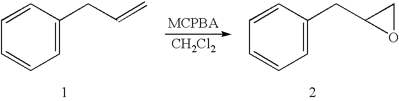

A 12 L four necked round bottom flask was equipped with a mechanical stirrer. The flask was charged with allyl benzene 1 (472 g, 4.0 mol) and dichloromethane (1 L) m-chloroperbenzoic acid (500 g, 2.9 mol) in dichloromethane (3 L) was added to the mixture in portions over 1.5 h (internal temperature kept below 35° C., the initial exotherm was controlled with an ice bath). A slurry of m-chloroperbenzoic acid (400 g, 2.3 mol) in dichloromethane (3 L) was added to the reaction in portions over 1 h. The reaction was allowed to warm to room temperature and m-chloroperbenzoic acid (114 g, 0.66 mol) was added to the reaction as a solid. The reaction was left stirring overnight. The following morning analysis by gas chromatography (GC) showed the reaction was 98% complete. To drive the reaction to completion m-chloroperbenzoic acid (50 g, 0.29 mol) was added to the reaction as a solid. After 5 h of stirring the reaction showed a conversion of 99.1% by GC. The reaction was filtered to remove solids and the filtrate was concentrated under reduced pressure. Additional solid formed upon concentration and was removed by filtration. Tert-Butyl methyl ether (1 L) was added to the filtrate. The solution was washed with a NaHCO₃ solution (4×300 mL), a Na₂SO₃ solution (50 g/300 mL H₂O), and then a Na₂SO₃ solution (80 g/300 mL H₂O) by stirring for 1.5 h. The organic layer was separated, dried over Na₂SO₄ and concentrated under reduced pressure. Vacuum distillation afforded 402.0 g of (2,3-epoxypropyl)benzene 2 for an isolated yield of 75%.

### B. Preparation of (R)-(2,3-epoxypropyl)benzene

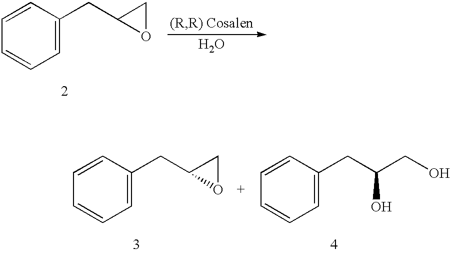

A 250 mL three necked round bottom flask was fitted with a mechanical stirrer. To the flask was added (R,R) Jacobsen Cobalt Catalyst (1.1 g, 1.9 mmol), (2,3-epoxypropyl)benzene 2 (50 g, 0.37 mol), acetic acid (0.46 g, 7.6 mmol), and tetrahyrofuran (3.9 mL, 3.5 g, 49 mmol). The mixture was cooled in an ice bath as water (3.6 mL, 0.20 mol) was added slowly. The reaction mixture was allowed to warm to room temperature and was left to stir overnight.

The following morning chiral GC analysis showed the reaction to be 95.6% complete. To drive the reaction to completion water (0.32 mL, 0.02 mol) was added. After 2 h of stirring, chiral GC analysis showed the reaction to be 97.6% complete. The reaction mixture was distilled at 1 torr and the product was collected at 55° C. (R)-(2,3-epoxypropyl)benzene 3 (98% ee, >99% chemical purity by GC) weighed 15 g for an isolated yield of 30%.

### C. Preparation of (S)-benzylisobutylcarbinol

### D.

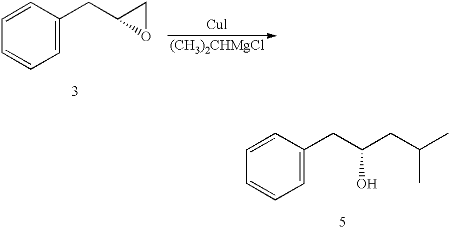

A dried 250 mL three necked round bottom flask was equipped with a mechanical stirrer, thermocouple, and a Claisen adapter fitted with a dropping funnel and rubber septum. To the flask was added Copper (I) iodide (1.0 g, 5.5 mmol). The flask was flushed with nitrogen and a nitrogen atmosphere was maintained thought the remaining steps. A 2.0 M solution of isopropyl magnesium chloride in tetrahydrofuran (65 mL, 13.7 g, 0.13 mol) was added and the mixture was stirred at room temperature for 0.5 h. The mixture was cooled to −60° C. in a dry ice/IPA bath. (R)-(2,3-epoxypropyl)benzene 3 (15.0 g, 0.11 mol) in anhydrous tetrahydrofuran (60 mL) was added drop wise slowly to avoid exotherm. Following the completion of addition the reaction mixture was allowed to stir at −60° C. for 0.5 h. The reaction mixture was slowly allowed to warm to room temperature. A latent exotherm was noted. The reaction mixture was cooled in an ice bath and the reaction was quenched with a 25% solution of NH₄Cl in water. The mixture was poured into a separatory funnel. Additional water was added and the product was extracted with ethyl acetate (2×). The combined organic layers were washed with brine, dried with Na₂SO₄, and concentrated. The crude product weighed 19.1 g. The product was distilled at 1 torr and collected at 83° C. (S)-Benzylisobutylcarbinol 5 (97.8% purity by GC) weighed 14.0 g for a isolated yield of 72%. The (S)-benzylisobutylcarbinol product exhibited a particularly interesting floral-green, mimosa powdery note. with a very natural effect.

### EXAMPLE 2

### Preparation of (R)-4-(2-Methyl-1-pentenyl) isobutyrate

### A. Preparation of (R)-4-Hydroxy-2-methyl-1-pentene

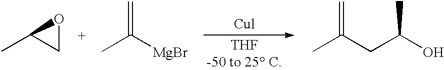

A dried 4-neck 1L, round bottomed flask equipped with a reflux condenser, an inlet temperature well, a mechanical stirrer and an addition funnel was charged with magnesium turnings (6 g; 247 mmol). The flask was heated with a heat gun while under a positive pressure of nitrogen. Upon cooling, THF (100 mL) was charged followed by a 10% of the total volume of a solution of 2-bromopropene (31.2 g; 258 mmol) in THF (100 mL). The remaining 2-bromopropene solution was then added dropwise keeping the internal temperature between 59-61° C. The resulting dark brown mixture, at which point there was no evidence of unreacted magnesium turnings, was allowed to cool to ambient temperature and charged with copper(I) iodide (1.6 g; 8.4 mmol). Subsequently, the reaction vessel was cooled to −50° C. and a solution of (R)-propylene oxide (5 g; 86.1 mmol, 99% ee) in THF (12 mL) was slowly added dropwise between the temperature ranges of −53 to −49° C. The resulting mixture was stirred at −50° C. for 0.5 h and then allowed to warm to ambient temperature where it was kept stirring overnight. After GC analysis of an aliquot revealed no unreacted propylene oxide, the reaction vessel was placed in an ice bath and slowly quenched with saturated aqueous solution of ammonium chloride (NH₄Cl). The heterogeneous mixture was filtered through a pad of celite and the filtrate was extracted with diethyl ether (100 mL). The organic layer was then washed with brine solution (3×100 mL), dried over anhydrous magnesium sulfate, filtered and the removal of the organic solvent furnished 9.48 g of crude yellow oil. The crude oil was distilled to provide 4.52 g (55%) of the title compound as a colorless oil, which was found to have a 95% purity (GC, % area) and 98% ee (as the trifluoroacetate derivative).

### B. Preparation of (R)-4-(2-Methyl-1-pentenyl) isobutyrate

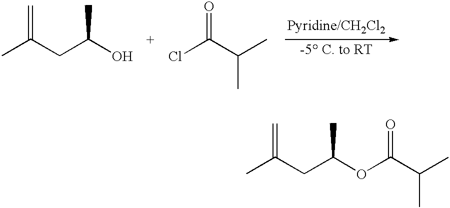

To a cold (ice/water/NaCl) mixture of (R)-4-hydroxy-2-methyl-1-pentene (10 g; 99.8 mmol), pyridine (16 mL; 198 mmol) in methylene chloride (70 mL) was added neat isobutyryl chloride (16 mL; 153 mmol) in portions. The resulting heterogeneous mixture was stirred in the cold bath for an additional 15 minutes and then stirred at ambient temperature. GC analysis of an aliquot from the reaction mixture revealed total conversion of the alcohol to the expected isobutyrate in 1.5 h. The mixture was extracted with 10% aqueous HCl (2×100 mL), saturated aqueous NaHCO₃ (2×100 mL) and then with brine (100 mL). The organic layer was dried over anhydrous sodium sulfate, filtered and concentrated to furnish 18.85 g of a yellow-orange oil. The oil was contaminated with the starting isobutyryl chloride, which was then treated with pyridine (5 mL) and methanol (9 mL). Again, the mixture was taken up in hexanes (100 mL) and washed with 10% aqueous HCl (2×100 mL), brine (100 mL), and the organic was dried over anhydrous sodium sulfate, filtered and concentrated to furnish a yellow oil. Distillation afforded 7.65 g (45%), which was observed to be of 97% purity (GC, % area), of the title ester as a colorless oil. The (R)-4-(2-methyl-1-pentenyl) isobutyrate product exhibited a fruity, apricot scent, with rosy, bay leaf and butyric notes.

### EXAMPLE 3

### Preparation of (S)-4-(2-Methyl-1-pentenyl) isobutyrate

### A. Preparation of (S)-4-Hydroxy-2-methyl-1-pentene

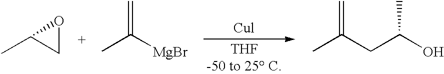

A dried 4-neck 1L, round bottomed flask equipped with a reflux condenser, an inlet temperature well, a mechanical stirrer and an addition funnel was charged with magnesium turnings (3.1 g; 128 mmol). The flask was heated with a heat gun while under a positive pressure of nitrogen. Upon cooling, THF (50 mL) was charged followed by a 10% of the total volume of a solution of 2-bromopropene (16.3 g; 135 mmol) in THF (50 mL). The remaining 2-bromopropene solution was then added dropwise keeping the internal temperature between 59-61° C. The resulting dark brown mixture, at which point there was no evidence of unreacted magnesium turnings, was allowed to cool to ambient temperature and charged with a 0.5 M solution of isopropenylmagnesium bromide (246 mL; 123 mmol) and copper(I) iodide (1.6 g; 8.4 mmol). Subsequently, the reaction vessel was cooled to −50° C. and a solution of (S)-propylene oxide (5 g; 86.1 mmol, 99% ee) THF (12 mL) was slowly added dropwise between the temperature ranges of −53 to −49° C. The resulting mixture was stirred at −50° C. for 0.5 h and then allowed to warm to ambient temperature where it was kept stirring overnight. After GC analysis of an aliquot revealed no unreacted propylene oxide, the reaction vessel was placed in an ice bath and slowly quenched with saturated aqueous solution of ammonium chloride (NH₄Cl). The heterogeneous mixture was filtered through a pad of celite and the filtrate was extracted with diethyl ether (100 mL). The organic layer was then washed with brine solution (3×100 mL), dried over anhydrous magnesium sulfate, filtered and the removal of the organic solvent furnished a crude yellow oil. The crude oil was distilled to provide 3.51 g (43%) of the title compound as a colorless oil, which was found to have a 90% purity (GC, % area) and 99% ee (as the trifluoroacetate derivative).

### B. Preparation of (S)-4-(2-Methyl-1-pentenyl) isobutyrate

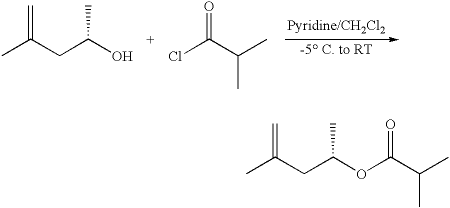

To a cold (ice/water/NaCl) mixture of (S)-4-hydroxy-2-methyl-1-pentene (10 g; 99.8 mmol), pyridine (16 mL; 198 mmol) in methylene chloride (70 mL) was added neat isobutyryl chloride (16 mL; 153 mmol) in portions. The resulting heterogeneous mixture was stirred in the cold bath for an additional 15 minutes and then stirred at ambient temperature. GC analysis of an aliquot from the reaction mixture revealed total conversion of the alcohol to the expected isobutyrate in 1.5 h. The mixture was extracted with 10% aqueous HCl (2×100 mL), saturated aqueous NaHCO₃ (2×100 mL) and then with brine (100 mL). The organic layer was dried over anhydrous sodium sulfate, filtered and concentrated to furnish 18.64 g of a yellow-orange oil. The oil was contaminated with the starting isobutyryl chloride, which was then treated with pyridine (5 mL) and methanol (9 mL). Again, the mixture was taken up in hexanes (100 mL) and washed with 10% aqueous HCl (2×100 mL), brine (100 mL), and the organic was dried over anhydrous sodium sulfate, filtered and concentrated to furnish a yellow oil. Distillation afforded 7.93 g (47%), which was observed to be of 96% purity (GC, % area), of the title ester as a colorless oil. The (s)-4-(2-methyl-1-pentenyl) isobutyrate product exhibited a prune scent, with mossy, camphorous and butyric notes.

### EXAMPLE 4

### Preparation of (S)-3-Hydroxyheptane

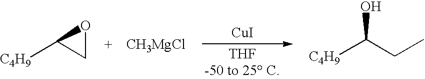

A dried 4-neck 1L, round bottomed flask equipped with a reflux condenser, an inlet temperature well, a mechanical stirrer and an addition funnel was charged with copper(I) iodide (1.8 g; 9.5 mmol). The flask was then purged with positive flow of nitrogen followed by the addition of a 3 M solution of methylmagnesium chloride (40 mL; 120 mmol) in THF in one portion and additional THF (100 mL). Subsequently, the reaction vessel was cooled to −50° C. and a solution of (R)-1,2-epoxyhexane (10 g; 99 mmol, 99% ee) THF (40 mL) was slowly added dropwise between the temperature range of −52 to −49° C. The resulting mixture was stirred at the above temperature range for 0.5 h and allowed to warm to ambient temperature, where it remained stirring overnight. GC analysis of an aliquot of the reaction mixture revealed no starting epoxide and the reaction vessel was placed in an ice bath followed by slow quenching with saturated aqueous ammonium chloride (NH₄Cl). The resulting heterogeneous mixture was then filtered through a pad of celite and the filtrate was taken up in diethyl ether. The organic layer was washed with brine solution, dried over anhydrous magnesium sulfate, filtered and the volatiles were removed under reduced pressure to furnish 11.47 g of crude yellow oil. The yellow oil was distilled to afford 7.21 g (63%) of the title alcohol as a colorless oil in 99% ee and observed to be of 99% purity (GC, % area). The (S)-3-hydroxyheptane product exhibited a lavender medicinal scent.

### EXAMPLE 5

### Preparation of (R)-3-Hydroxyheptane

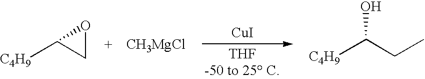

A dried 4-neck 1L, round bottomed flask equipped with a reflux condenser, an inlet temperature well, a mechanical stirrer and an addition funnel was charged with copper(I) iodide (1.8 g; 9.5 mmol). The flask was then purged with positive flow of nitrogen followed by the addition of a 3 M solution of methylmagnesium chloride (40 mL; 120 mmol) in THF in one portion and additional THF (100 mL). Subsequently, the reaction vessel was cooled to −50° C. and a solution of (S)-1,2-epoxyhexane (10 g; 99 mmol, 99% ee) in THF (40 mL) was slowly added dropwise between the temperature range of −52 to −49° C. The resulting mixture was stirred at the above temperature range for 0.5 h and allowed to warm to ambient temperature, where it remained stirring overnight. GC analysis of an aliquot of the reaction mixture revealed no starting epoxide and the reaction vessel was placed in an ice bath followed by slow quenching with saturated aqueous ammonium chloride (NH₄Cl). The resulting heterogeneous mixture was then filtered through a pad of celite and the filtrate was taken up in diethyl ether. The organic layer was washed with brine solution, dried over anhydrous magnesium sulfate, filtered and the volatiles were removed under reduced pressure to furnish 10.1 g of crude yellow oil. The yellow oil was distilled to afford 7.56 g (66%) of the title alcohol as a colorless oil in 99% ee and observed to be of 99% purity (GC, % area). The of (R)-3-hydroxyheptane product exhibited an earthy, mushroom scent.

### EXAMPLE 6

### Preparation of (R)-2-Hydroxyheptane

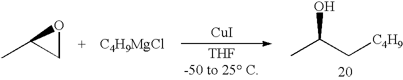

A dried 4-neck 1L, round bottomed flask equipped with a reflux condenser, an inlet temperature well, a mechanical stirrer and an addition funnel was charged with copper(I) iodide (4.8 g; 25 mmol). The flask was then purged with positive flow of nitrogen followed by the addition of a 2 M solution of n-butylmagnesium chloride (155 mL; 310 mmol) in THF in one portion and additional THF (100 mL). Subsequently, the reaction vessel was cooled to −50° C. and a solution of (R)-propylene oxide (15 g; 258 mmol, 99% ee) in THF (50 mL) was slowly added dropwise between the temperature ranges of −53 to −49° C. The resulting mixture was stirred at the above temperature range for 0.5 h and allowed to warm to ambient temperature, where it remained stirring overnight. GC analysis of an aliquot of the reaction mixture revealed no starting epoxide and the reaction vessel was placed in an ice bath followed by slow quenching with saturated aqueous ammonium chloride (NH₄Cl). The resulting heterogeneous mixture was then extracted with ether and brine. The organic was dried over anhydrous sodium sulfate, filtered and concentrated via reduced pressure to furnish 27.06 g of crude yellow oil. The yellow oil was distilled (147-152° C.) to afford 18.98 g (63%) of the title alcohol as a colorless oil in 99% ee and observed to be of 99% purity (GC, % area). The (R)-2-hydroxyheptane product exhibited a lavender scent.

### EXAMPLE 7

### Preparation of (S)-2-Hydroxyheptane

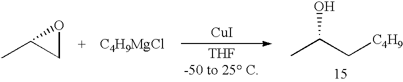

A dried 4-neck 1L, round bottomed flask equipped with a reflux condenser, an inlet temperature well, a mechanical stirrer and an addition funnel was charged with copper(I) iodide (4.8 g; 25 mmol). The flask was then purged with positive flow of nitrogen followed by the addition of a 2 M solution of n-butylmagnesium chloride (155 mL; 310 mmol) in THF in one portion and additional THF (100 mL). Subsequently, the reaction vessel was cooled to −50° C. and a solution of (S)-propylene oxide (15 g; 258 mmol, 99% ee) in THF (50 mL) was slowly added dropwise between the temperature ranges of −53 to 49° C. The resulting mixture was stirred at the above temperature range for 0.5 h and allowed to warm to ambient temperature, where it remained stirring overnight. GC analysis of an aliquot of the reaction mixture revealed no starting epoxide and the reaction vessel was placed in an ice bath followed by slow quenching with saturated aqueous ammonium chloride (NH₄Cl). The resulting heterogeneous mixture was then extracted with ether and brine. The organic was dried over anhydrous sodium sulfate, filtered and concentrated via reduced pressure to furnish 30.28 g of crude yellow oil. The yellow oil was distilled to afford 22.04 g (73%) of the title alcohol as a colorless oil in 99% ee and observed to be of 99% purity (GC, % area). The (S)-2-hydroxyheptane product exhibited a lavender scent.

### EXAMPLE 8

### Preparation of (S)-3-Acetoxyheptane

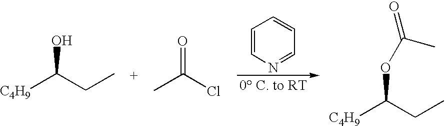

To a cold (ice/water) mixture of (S)-3-hydroxyheptane (4.97 g; 42.8 mmol) in pyridine (40 mL; 495 mmol) was added dropwise neat acetyl chloride (6.1 mL; 85.6 mmol). The resulting heterogeneous mixture was stirred at ambient temperature overnight. The mixture was then partitioned between water and diethyl ether. The organic layer was washed several times with 10% HCl (aq.), dried over anhydrous sodium sulfate, filtered and concentrated to furnish 6.83 g of a yellow-orange oil. Two distillations were performed to afford 4.11 g (61%), which was observed to be of 99% purity (GC, % area) of the title ester (150-155° C.) as a colorless oil. The (S)-3-acetoxyheptane product exhibited a rosy, fresh, agrest scent.

### EXAMPLE 9

### Preparation of (R)-3-Acetoxyheptane

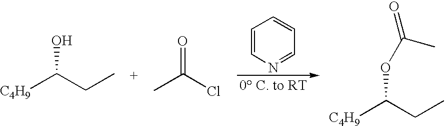

To a cold (ice/water) mixture of (R)-3-hydroxyheptane (3.77 g; 32.4 mmol) in pyridine (30 mL; 495 mmol) was added dropwise neat acetyl chloride (4.6 mL; 64.8 mmol). The resulting heterogeneous mixture was stirred at ambient temperature overnight. The mixture was then partitioned between water and diethyl ether. The organic layer was washed several times with 10% HCl (aq.), dried over anhydrous sodium sulfate, filtered and concentrated to furnish 4.85 g of a yellow-orange oil. Two distillations were performed to afford 3.14 g (61%), which was observed to be of 99% purity (GC, % area), of the title ester as a colorless oil. The (R)-3-acetoxyheptane product exhibited a green, fruity pear scent.

### EXAMPLE 10

### Preparation of (R)-2-Acetoxyheptane

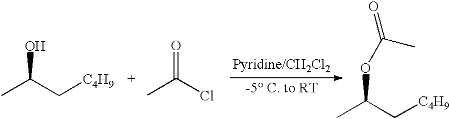

To a cold (ice/water/NaCl) mixture of (R)-2-hydroxyheptane (10.7 g; 92 mmol), pyridine (14.9 mL; 184 mmol) in methylene chloride (50 mL) was added neat acetyl chloride (9.8 mL; 138 mmol) in portions. The resulting heterogeneous mixture was stirred in the cold bath for an additional 15 minutes and then stirred at ambient temperature overnight. GC analysis of an aliquot from the reaction mixture revealed total conversion of the alcohol to the expected acetate. The mixture was extracted with aqueous 10% HCl (3×100 mL) and then with brine (100 mL). The organic layer was dried over anhydrous sodium sulfate, filtered and concentrated to furnish 13.81 g of a yellow-orange oil. Two distillations were performed to afford 9.87 g (68%), which was observed to be of 99% purity, of the title ester as a colorless oil. The (R)-2-acetoxyheptane product exhibited a fruity, verveine scent.

### EXAMPLE 11

### Preparation of (S)-2-Acetoxyheptane

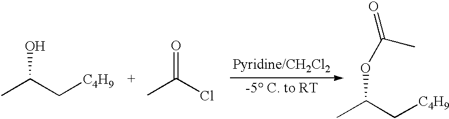

To a cold (ice/water/NaCl) mixture of (R)-2-hydroxyheptane (10.7 g; 92 mmol), pyridine (14.9 mL; 184 mmol) in methylene chloride (50 mL) was added neat acetyl chloride (9.8 mL; 138 mmol) in portions. The resulting heterogeneous mixture was stirred in the cold bath for an additional 15 minutes and then stirred at ambient temperature overnight. GC analysis of an aliquot from the reaction mixture revealed total conversion of the alcohol to the expected acetate. The mixture was extracted with aqueous 10% HCl (3×100 mL) and then with brine (100 mL). The organic layer was dried over anhydrous sodium sulfate, filtered and concentrated to furnish 14.49 g of a yellow-orange oil. Two distillations were performed to afford 10.54 g (72%), which was observed to be of 99% purity, of the title ester as a colorless oil. The (S)-2-acetoxyheptane product exhibited a green, fruity, chemical scent.

### EXAMPLE 12

### Preparation of (R)-Okoumal

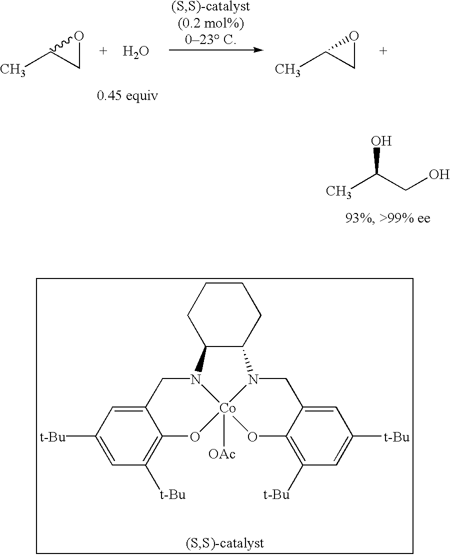

(R)-Propanediol. A 3-neck 250-mL round bottomed flask equipped with a large magnetic stirbar was charged with (S,S)-Co(salen) catalyst (1.21 g, 2 mmol, 0.2 mol %), CH₂Cl₂ (10 mL), and glacial acetic acid (0.23 mL, 4 mmol, 2 equiv to catalyst). The red heterogeneous mixture was stirred open to air for 2 hours, during which time the mixture changed to a dark brown, homogeneous solution. To the mixture was added racemic propylene oxide (70.7 mL, 1.0 mol), and the resulting brown mixture was cooled in an ice bath. Water addition was begun in 0.5 mL increments via syringe (8.1 mL total, 0.45 mol, 0.45 equiv to epoxide) in order to maintain the reaction temperature below 15° C. (addition is exothermic). After complete addition, the ice bath was allowed to expire, and the mixture was stirred for a total of 8-10 hours. At this point, the flask was affixed with a distillation head, and the unreacted epoxide and CH₂Cl₂ were removed by distillation under N₂. The receiving flask was exchanged, and the red heterogeneous mixture was placed under vacuum (2-5 mm Hg). The (R)-1,2-propanediol was distilled under vacuum to yield 31.7 g (0.42 mol, 93% of theory based on water added) as a clear, colorless liquid. Analysis of the product by chiral GC (of the bis(trifluoroacetate) derivative) showed the product to be 99.4% ee.

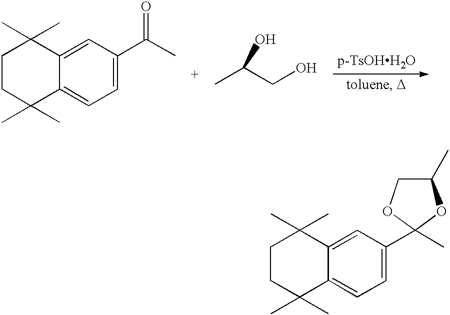

(R)-Okoumal. To a solution of the starting ketone (8 g; 34.7 mmol) and (R)-1,2-propanediol (6.61 g; 86.8 mmol) in toluene (40 mL) was added p-toluenesulfonic acid monohydrate (80 mg; 0.421 mmol). The resulting mixture was heated to reflux for ca. 3 hours. The mixture was partitioned between toluene and saturated aqueous NaHCO₃. The organic extract was dried over anhydrous sodium sulfate, filtered and concentrated to furnish 8.94 g of a crude viscous oil. The oil was distilled at 128-129° C. (1 torr) to afford 7.41 g (74%) of the title Okoumal as a diastereomeric mixture in 97% purity (GC, % area). The (R)-Okoumal exhibited an amber, fresh, sweet, powdery, slightly musky, dull scent.

### EXAMPLE 13

### Preparation of (S)-Okoumal

(S)-1,2-propanediol was produced using (R,R)-Co(salen) catalyst in a manner analogous to that disclosed above for (R)-propanediol.

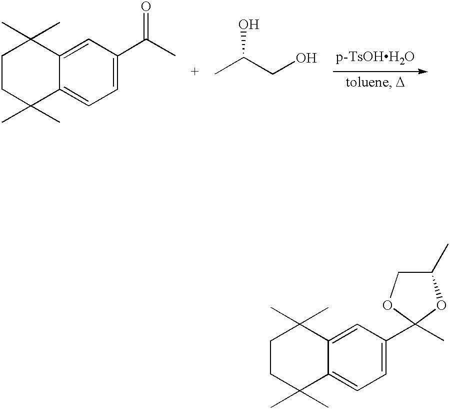

(S)-Okuomal. To a solution of the starting ketone (8 g; 34.7 mmol) and (S)-1,2-propanediol (6.61 g; 86.8 mmol) in toluene (40 mL) was added p-toluenesulfonic acid monohydrate (80 mg; 0.421 mmol). The resulting mixture was heated to reflux for ca. 3 hours. The mixture was partitioned between toluene and saturated aqueous NaHCO₃. The organic extract was dried over anhydrous sodium sulfate, filtered and concentrated to furnish 8.94 g of a crude viscous oil. The oil was distilled at 128-129° C. (1 torr) to afford 7.79 g (78%) of the title Okoumal as a diastereomeric mixture in 99% purity (GC, % area). The (S)-Okoumal exhibited an amber, woody, fresh cut Paris mushrooms, wood glue, slightly pungent scent.

The present invention has been described with particular reference to the preferred embodiments. It should be understood that the foregoing descriptions and examples are only illustrative of the invention. Various alternatives and modifications thereof can be devised by those skilled in the art without departing from the spirit and scope of the present invention. Accordingly, the present invention is intended to embrace all such alternatives, modifications, and variations that fall within the scope of the appended claims.

What is claimed is:

1. A process for enantioselective preparation of a non-racemic compound usable as a fragrance or flavor component or is convertible to a fragrance or flavor component, said process comprising contacting:
a substrate capable of forming a non-racemic compound by an enantioselective reaction and at least one co-reactant in the presence of a non-racemic catalyst; or
a non-racemic or enantiopure substrate and at least one co-reactant, optionally in the presence of a racemic or non-racemic catalyst;
said contacting being at a temperature and for a length of time sufficient to produce said non-racemic compound.

2. The process of claim 1, wherein said non-racemic compound is selected from the group consisting of an enantiopure enantiomer or an enantiomerically enriched mixture of enantiomers represented by the formulae, wherein chiral centers are indicated by an asterisk:
R₁*CHR₂R₃ (1 and 2), wherein 1 and 2 are represented by the formulae: 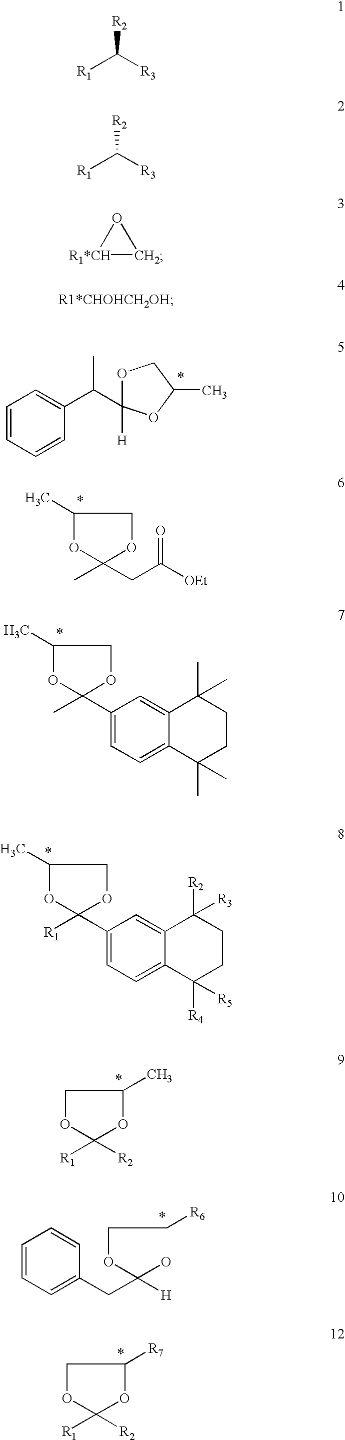
wherein R₁ in compounds 1 and 2 is selected from the group consisting of: a linear, branched or cyclic alkyl of 1 to 12 carbon atoms, aryl of 6 to 12 carbon atoms, aralkyl of 7 to 14 carbon atoms, alkaryl of 7 to 14 carbon atoms and ((1R, 2S, 5R)-(−)-menthoxy)CH₂; R₂ is selected from the group consisting of: hydroxy and a carboxylate of 1 to 12 carbon atoms; and R₃ is selected from the group consisting of: a linear, branched or cyclic alkyl of 1 to 12 carbon atoms, alkenyl of 1 to 12 carbon atoms, hydroxymethyl and acyloxymethyl of 1 to 12 carbon atoms;
wherein R₁ in compounds 3 and 4 is selected from the group consisting of: a linear, branched or cyclic alkyl of 1 to 12 carbon atoms, aryl of 6 to 12 carbon atoms, aralkyl of 7 to 14 carbon atoms, alkaryl of 7 to 14 carbon atoms, halomethyl and ((1R, 2S, 5R)-(−)-menthoxy)CH₂;
wherein each R₁, R₂, R₃, R4 and R₅ in compound 8 is independently selected from the group consisting of: hydrogen, a linear, branched or cyclic alkyl of 1 to 12 carbon atoms, aryl of 6 to 12 carbon atoms, aralkyl of 7 to 14 carbon atoms and alkaryl of 7 to 14 carbon atoms;
wherein each R₁ and R₂ in compound 9 is independently selected from the group consisting of: hydrogen, a linear, branched or cyclic alkyl of 1 to 12 carbon atoms, aryl of 6 to 12 carbon atoms, aralkyl of 7 to 14 carbon atoms and alkaryl of 7 to 14 carbon atoms;
wherein R₆ in compound 10 is hydroxymethyl;
wherein each R₁ and R₂ in compound 12 is selected from the group consisting of: a linear, branched or cyclic alkyl of 1 to 12 carbon atoms, aryl of 6 to 12 carbon atoms, aralkyl of 7 to 14 carbon atoms and alkaryl of 7 to 14 carbon atoms; and R₇ is selected from the group consisting of: a linear, branched or cyclic alkyl of 1 to 12 carbon atoms, aryl of 6 to 12 carbon atoms, heteroaryl of 6 to 12 carbon atoms, aralkyl of 7 to 14 carbon atoms and alkaryl of 7 to 14 carbon atoms; and
wherein R₁ in compound 13 is phenyl; R₂ is methyl; R₃ is hydrogen; and R₄ is CO₂Et.

3. The process of claim 1, wherein said non-racemic compound has an optical purity of at least 1% enantiomeric excess.

4. The process of claim 1, wherein said non-racemic compound has an optical purity of at least 75% enantiomeric excess.

5. The process of claim 1, wherein said non-racemic compound has an optical purity of at least 95% enantiomeric excess.

6. The process of claim 1, wherein said non-racemic compound has an optical purity of at least 99% enantiomeric excess.

7. The process of claim 1, wherein said non-racemic compound is an enantiopure single enantiomer.

8. The process of claim 1, wherein said enantioselective reaction is selected from the group consisting of: hydrogenation, hydroboration, hydride transfer, alkylation, vinylation, epoxidation, epoxide ring opening, acetalization, ketalization, acylation, nucleophilic substitution and a combination thereof.

9. The process of claim 8, wherein said enantioselective reaction is asymmetric epoxidation, said substrate capable of forming a non-racemic compound by an enantioselective reaction is 14a (R₁=phenyl; R₂=methyl; R₃═H; R₄═CO₂Et), said co-reactant is an epoxidation agent, said non-racemic catalyst is (R, R)-Mn Salen catalyst and said non-racemic compound is fragrance (R, R)- 13a (R₁=phenyl; R₂=methyl; R₃═H; R₄═CO₂Et) obtained in an optical purity of greater than 80% ee.

10. The process of claim 8, wherein said enantioselective reaction is asymmetric epoxidation, said substrate capable of forming a non-racemic compound by an enantioselective reaction is 14a (R₁=phenyl; R₂=methyl; R₃═H; R₄═CO₂Et), said co-reactant is an epoxidation agent, said non-racemic catalyst is (S, S)-Mn Salen catalyst and said non-racemic compound is fragrance (S, S)-13a (R₁=phenyl; R₂=methyl; R₃═H; R₄═CO₂Et) obtained in an optical purity of greater than 80% ee.

11. The process of claim 8, wherein said enantioselective reaction is epoxide ring opening under hydrolytic kinetic resolution conditions, said substrate capable of forming a non-racemic compound by an enantioselective reaction is racemic hexyl oxirane, said co-reactant is water, said non-racemic catalyst is (R, R)-Co Salen catalyst and said non-racemic compound is fragrance precursor (S)-octane-1,2-diol and (R)-hexyl oxirane, each obtained in an optical purity of greater than 95% ee.

12. The process of claim 8, wherein said enantioselective reaction is epoxide ring opening under hydrolytic kinetic resolution conditions, said substrate capable of forming a non-racemic compound by an enantioselective reaction is racemic hexyl oxirane, said co-reactant is water, said non-racemic catalyst is (S, S)-Co Salen catalyst and said non-racemic compound is fragrance precursor (R)-octane-1,2-diol and (S)-hexyl oxirane each obtained in an optical purity of greater than 95% ee.

13. The process of claim 8, wherein said enantioselective reaction is ketalization, said non-racemic or enantiopure substrate is (R)-propylene glycol (1 g) or (S)-propylene glycol (2 g), said co-reactant is dihexyl ketone, said catalyst is a ketalization catalyst, and said non-racemic compound is fragrance (R)- or (S)-enantiomer, of ketal 9a, respectively, wherein each enantiomer is obtained in an optical purity of greater than 98% ee.

14. The process of claim 8, wherein said enantioselective reaction is epoxide ring opening, said non-racemic or enantiopure substrate is (R)-hexyl oxirane or (S)-hexyl oxirane, said co-reactant is selected from the group consisting of: methyl magnesium halide and methyl lithium, and said non-racemic compound is fragrance (R)- or (S)-enantiomer of 3-nonanol (1a) and (2a), respectively, wherein each enantiomer is obtained in an optical purity of greater than 95% ee.

15. The non-racemic compound of claim 1, wherein said non-racemic compound is selected from the group consisting of: an enantiopure and an enantiomerically enriched compound.

16. A non-racemic compound, which is usable as a fragrance or flavor component or is convertible to a fragrance or flavor component, prepared by a process comprising contacting:
a substrate capable of forming a non-racemic compound by an enantioselective reaction and at least one co-reactant in the presence of a non-racemic catalyst; or a non-racemic or enantiopure substrate and at least one co-reactant, optionally in the presence of a racemic or non-racemic catalyst;
said contacting being at a temperature and for a length of time sufficient to produce said non-racemic compound.

17. A perfume comprising:
a non-racemic compound according to claim 16; and
at least one compound selected from the group consisting of: an alcohol, aldehyde, ketone, ester, lactone, acetal, indole, p-menthane-8-thiol-3-one, methyleugenol, eugenol, anethol, solvent and diluent.

18. A scented product selected from the group consisting of: eau de cologne, scented water, toilet water, cream, shampoo, deodorant, soap, detergent powder, household cleaner and softener, comprising a non-racemic compound according to claim 16.

19. A non-racemic composition comprising an enantiopure enantiomer of structural formula (1) or (2), or a non-racemic mixture of an enantiomer of structural formula (1) and an enantiomer of structural formula (2): 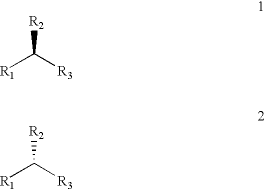
wherein:
R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;
R′ is (C₁-C₄)alkylene;
R″ is (C₁-C₁₂)alkyl, (C₆-C₁₂)aryl, (C₇-C₁₄)aralkyl, (C₇-C₁₄)alkaryl or (C₁-C₁₂)carboxylate; and
R₂ is hydroxy or (C₁-C₁₂)carboxylate.

20. A non-racemic composition according to claim 19, wherein R₁ is (C₇-C₁₄)aralkyl; R₂ is hydroxy or (C₁-C₁₂)carboxylate; and R₃ is (C₁-C₁₂)alkyl.

21. A non-racemic composition according to claim 19, wherein R₁ is benzyl, R₂ is hydroxy and R₃ is isobutyl.

22. A process for making of non-racemic composition according to claim 21, comprising:
reacting an optically active epoxy type reactant with formula: 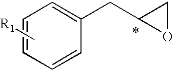
wherein R₁ is hydrogen, (C₁-C₄)alkyl or (C₁-C₄)alkoxy, and
a magnesium halide of the formula: 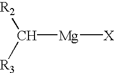
wherein R₂ and R₃, are each independently H, (C₁-C₁₂)alkyl, (C₂-C₁₂)alkenyl or (C₁-C₁₂)alkoxyalkyl and X is halo.

23. A process according to claim 22 wherein the reaction is carried out in the presence of a copper iodide or iodine catalyst.

24. A process according to claim 22 wherein in that (S)-benzylisobutylcarbinol, is obtained by reacting the magnesium halide with (R)-(2,3-epoxypropyl)benzene.

25. An optically active epoxide according to the formula: 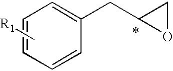
wherein R₁ is hydrogen, (C₁-C₄)alkyl or (C₁-C₄)alkoxy.

26. A process for making an optically active epoxide according to claim 25, comprising reacting a peracid with the unsaturated compound corresponding to formula: 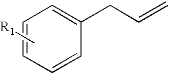
wherein R₁ is hydrogen, (C₁-C₄)alkyl or (C₁-C₄)alkoxy, to form a racemic epoxide mixture and subsequently splitting the racemic epoxide mixture by hydrolytic kinetic resolution to produce one enantiomer in the form of a di-alcohol and another enantiomer in the form of the optically active epoxide.

27. A process according to claim 26 wherein the hydrolytic kinetic resolution is carried out with water in the presence of an optically active catalyst.

28. A process according to claim 27 wherein the optically active catalyst is a complex between a transition metal, preferably Cr, Mn, V, Fe, Mo, W, Ru, Ni or Co, and the Salen ligand.

29. A process according to claim 27 wherein the optically active catalyst is the (R,R)-Co Salen catalyst.

30. A process for making a perfuming composition or perfumed article, comprising adding to such composition or article an effective quantity of a non-racemic composition comprising an enantiopure enantiomer of structural formula (1) or (2), or a non-racemic mixture of an enantiomer of structural formula (1) and an enantiomer of structural formula (2): 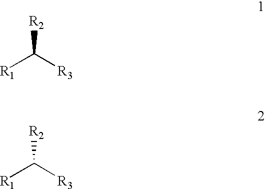
wherein:
R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;
R′ is (C₁-C₄)alkylene;
R″ is (C₁-C₁₂)alkyl, (C₆-C₁₂)aryl, (C₇-C₁₄)aralkyl, (C₇-C₁₄)alkaryl or (C₁-C₁₂)carboxylate; and
R₂ is hydroxy or (C₁-C₁₂)carboxylate.

31. A process according to claim 30, wherein the non-racemic composition is enatiopure (R) or (S)-benzylisobutylcarbinol enantiomer.

32. A process according to claim 31, wherein (S)-benzylisobutylcarbinol is used as a particularly interesting floral-green, mimosa, powdery fragrance note.

33. A process according to claim 31, wherein (R)-benzylisobutylcarbinol is used as a green rose fragrance note.

34. A perfuming composition or perfumed article, comprising an effective amount of a non-racemic composition comprising an enantiopure enantiomer of structural formula (1) or (2), or a non-racemic mixture of an enantiomer of structural formula (1) and an enantiomer of structural formula (2): 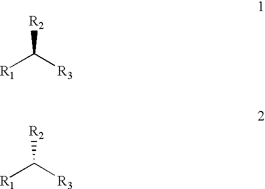
wherein:
R₁ and R₃ are each independently (C₁-C₁₂)alkyl, hydroxyalkyl, (C₂-C₁₂)alkenyl, (C₆-C₁₂)aryl, (C₇-C₁₄)aralkyl, (C₇-C₁₄)alkaryl, or R′OR″;
R′ is (C₁-C₄)alkylene;
R″ is (C₁-C₁₂)alkyl, (C₆-C₁₂)aryl, (C₇-C₁₄)aralkyl, (C₇-C₁₄)alkaryl or (C₁-C₁₂)carboxylate; and
R₂ is hydroxy or (C₁-C₁₂)carboxylate.

35, A perfuming composition or perfumed article according to claim 34, wherein the perfuming composition or perfumed article is in the form of a perfume, a eau de toilette, an after-shave lotion, a soap, a bath gel, a shower gel, a deodorant, an antiperspirant, a hair-care product, a powder, an air freshener, a cleaning product, a detergent composition or a fabric softener.

36. A composition according to claim 34, wherein the non-racemic composition is an enantiopure (R) or (S)-benzylisobutylcarbinol enantiomer.

37. A process for making a non-racemic compound usable as a fragrance or flavor component, comprising:
resolving a racemic epoxide by hydrolytic kinetic resolution to give a non-racemic epoxide and an non-racemic diol, and
condensing the non-racemic diol with a ketone or aldehyde to form the non-racemic compound.

38. The process of claim 37, wherein the racemic epoxide is racemic propylene oxide and the hydrolytic kinetic resolution is conducted with water in the presence of (S, S)-Co Salen catalyst and the non-racemic diol is (R)-propane-1,2-diol and the non-racemic epoxide is (S)-propylene oxide.

39. The process of claim 38, wherein the (R)-propane-1,2-diol and (S)-propylene oxide are each obtained in an optical purity of greater than 95% ee.

40. The process of claim 38, wherein the (R)-propane-1,2-diol is condensed with a ketone or aldehyde according to the structural formula: 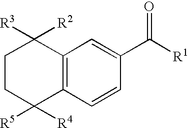
wherein R¹, R², R³, R⁴ and R⁵ are each independently H, alkyl or aryl,

41. The process of claim 40, wherein R¹, R², R³, R⁴ and R⁵ are each methyl and the non-racemic compound usable as a fragrance or flavor component is (R)-Okoumal.

42. The process of claim 37, wherein the racemic epoxide is racemic propylene oxide and the hydrolytic kinetic resolution is conducted with water in the presence of (R, R)-Co Salen catalyst, and the non-racemic diol is (S)-propane-1,2-diol and the non-racemic epoxide is (R)-propylene oxide.

43. The process of claim 42, wherein the (R)-propane-1,2-diol and (S)-propylene oxide are each obtained in an optical purity of greater than 95% ee.

44. The process of claim 42, wherein the (S)-propane-1,2-diol is condensed with a ketone or aldehyde according to the structural formula: 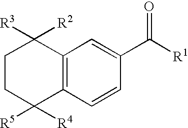
wherein R¹, R², R³, R⁴ and R⁵ are each independently H, alkyl or aryl.

45. The process of claim 43, wherein R¹, R², R³, R⁴ and R⁵ are each methyl and the non-racemic compound usable as a fragrance or flavor component is (S)-Okoumal.

46. A non-racemic compound made by the process of claim 37.

47. A process for making a perfuming composition or perfumed article, comprising adding to such composition or article an effective quantity of a non-racemic compound made by the process of claim 37.

48. A perfume composition of a perfumed article comprising a non-racemic compound made by the process of claim 37.

49. A non-racemic composition comprising an enantiopure (S)-Okoumal, enantiopure (R)-Okoumal or a non-racemic mixture of (S)-Okoumal and (R)-Okoumal.
